(19) United States
(12) Patent Application Publication
(10) Pub. No.: US 2003/0162704 A1
(43) Pub. Date: Aug. 28, 2003

(54) Method of treatment of parkison's disease with a protein extractable from mammalian organs

(76) Inventor: Alberto Panerai, Milano (IT)

Correspondence Address: NIXON & VANDERHYE, PC, 1100 N GLEBE ROAD, 8TH FLOOR, ARLINGTON, VA, 22201-4714, US

(21) Appl. No.: 10/297,670

(22) PCT Filed: Jun. 4, 2001
(86) PCT No.: PCT/EP01/06337

(51) Int. Cl. A61K 38/17

(52) U.S. Cl. 514/12

(57) ABSTRACT

A method of treatment of patients affected by Parkinson's disease comprising the administration of an effective amount of a 14 kDa protein extractable from mammalian organs, particularly mammalian liver.

The present invention concerns a method of treatment of patients affected by Parkinson's disease comprising the administration of an effective amount of a 14 kDa protein extractable from mammalian organs, particularly mammalian liver.

Parkinson's disease (incidence of 168/100,000) is a well-known, widespread disease, especially in the population of age above 60 years, and is characterized by impaired control of movement.

Parkinson's disease (PD) is a progressive degenerative disorder that results in incapacity to coordinate motor functions and lack of ability to care for themselves in patients within 10-15 years since diagnosis.

Several biochemical and genetic factors seem to be involved in the pathogenesis of PD, which remains however to be still elucidated. The formation of agglomerates of proteins in some brain areas of affected patients, to form the so-called Lewi's bodies, seems to be anyhow one of the main causes of the neurodegenerative effects typical of this disease.

Although the etiology of PD is still largely unknown, according to a recent hypothesis, the first step in the onset of PD appears to be connected with an increase of toxic factors such as oxygen radicals and the cited formation of protein agglomerates whereas the degenerative and progressive phase would seem to be at least partially activated and sustained by autoimmune mechanisms.

The presently available therapy based on Levodopa does not cure the pathology, but only reduces symptoms for a limited time, nor prevents the concomitant mental degeneration. Furthermore, such therapy often involves alterations of sleep and humoral behavior, thus making relations with people dealing with PD patients difficult.

Several experimental therapies have also been proposed, none of which seems however until now particularly promising.

One of the major difficulties in developing an effective treatment for PD is due to the lack of a reliable and predictive animal model so that the only definitive evidence on the actual effectiveness of a new therapy has to be obtained from clinical tests.

It has now been found that PD can be effectively treated by administering to affected patients a 14 kDa protein which is normally present in mammalian liver, particularly in goat liver, and which can be prepared either by extraction or by recombinant DNA methods.

Said protein, hereinafter referred to with the abbreviation of MFP 14 (derived from Multiple Function Protein 14 kDa) has been disclosed by Ceciliani et al., FEBS Lett., 1996;393;147-50.

Its cytotoxic activity has been reported in Int.J.Oncol., 1996; 8:543-48 whereas its cDNA and expression in E.coli is reported by Colombo et al. in Biochem. Biophys. Acta, 1998;1442:49-59.

The preparation of the extractive protein as well as the preparation of the recombinant protein have been respectively disclosed in U.S. Pat. No. 5,792,744 and in PCT/EP/00 03003 which are herein incorporated by reference.

Therapeutic uses of this protein in the treatment of AIDS, autoimmune disease and TNF-induced disease have been disclosed in WO 98/42366.

Moreover, said protein has been found to be an inhibitor of protein synthesis, a modulator of cytokines synthesis as well as specific calpain activator.

MFP 14 has some sequence similarities with Heat shock proteins or HSP, with the protein binding to the Major Histocompatibilty Complex-1 (MHC-1 binding protein) and with the YER057C/YIL051C/Y5GF family of proteins having a still unknown function, highly evolutionary conserved from prokaryotes to mammals.

The invention, according to a first embodiment, provides therefore a method of treatment of PD comprising the administration to patients in need of such treatment of a therapeutically active dose of MFP 14 or active fragment.

The invention also provides pharmaceutical compositions useful for treating Parkinson's disease containing as the active component an MFP 14 protein or active fragment.

The term MFP 14 refers also to proteins having high degree of homology with the amino acid sequence disclosed in the above-cited references. By high degree of homology, proteins having at least 70% homology with the 137 amino acid sequence of the native protein are meant. Preferably, the degree of homology is higher than 80%, more preferably higher than 90%.

The term “active fragment” refers to shorter sequences derived from the native or recombinant MFP 14 protein and still retaining the pharmacological activity of the parent sequence. It is in fact known that the therapeutic activity of a given protein does not always require a complete sequence, the activity being often confined to smaller regions, e.g. to N-terminal, Carboxy-terminal or internal regions. In such an event, it may be advantageous the administration of the active fragment rather than the intact protein in view of lower production costs, higher metabolic stability and other possible advantages connected with the administration of polypeptides having lower molecular weight.

The fragments and homologues of MFP 14 may also derive from deletion, substitutions and/or insertion mutation of amino acids. For instance, it is known that the so called “conservative” mutations, i.e. the substitution of an amino acid with another one of the same category (acidic, basic, neutral, hydrophilic or lipophilic), is usually acceptable for the preservation of activity.

The use of recombinant MFP 14 is particularly preferred in view of the easier availability and standardization of production methods.

Alternatively, an extract comprising MFP 14, such as that disclosed in WO 92/10197, may also be used.

For the considered therapeutic use, MFP 14 or active fragments thereof will be administered parenterally, e.g. by intramuscular or subcutaneous route, in form of sterile solutions or suspensions in acceptable carriers such as saline solutions, oils for parenteral administration and the like.

Other administration routes can also be envisaged, for instance the oral or transdermal route, using known methods for the delivery of proteins or polypeptides by these routes (e.g. by means of liposomes or micro-encapsulation methods).

The administration of MFP 14 proteins could also be carried out using gene therapy protocols, for instance by administering suitable vectors, which may deliver to target cells a gene sequence coding for MFP 14. Suitable vectors as well as corresponding control sequences and protocols are disclosed in FASEB J. 9, 190-199, 1995 and in Nature 392 (suppl. April 30) 25-30, 1998.

MFP 14 dose range which was found to be effective in the treatment of Parkinson's disease is comprised from about 1 mg to 10 mg/day.

The dose can be divided in more than one daily administration, for instance two or three administrations. In particular cases, the administrations can also be separated one from the other by longer period of times, up to 1-4 weeks. This can particularly apply to the chronic long-term treatment, once the first cycle of treatment has been completed.

The dosage regimen can anyhow vary within wide limits, in view of the very low toxicity of MFP 14, so that the skilled physicians will easily adapt the doses according to individual patients' requirements, particularly taking into consideration the age, sex, weight of the patient and the seriousness and advancement stage of the disease.

It has also been found that the administration of ubiquitin in combination with MFP 14 is advantageous in the treatment of PD. Ubiquitins belong to a well known family of proteins, the use of which has been proposed for several pathologies which do not have anything in common with PD. For the considered therapeutic use, ubiquitins will be administered, preferably contemporaneously, together with MFP 14, at a dosage ranging from about 1 mg to 10 mg/day.

According to a further embodiment, the invention provides therefore also pharmaceutical compositions comprising as the active ingredient a combination of MFP 14 and of ubiquitin, in admixture with a suitable pharmaceutical carrier.

The administration of MFP 14 or of fragments thereof, optionally in combination with ubiquitin, proved to be effective in clinical trials carried out on patients with PD at different stages. In particular the treatment of the invention turned out to be effective both in the first stages as well as in the late stages of this pathology, inducing a significant recovery of the motion function and an improvement of the social life in affected patients.

The following examples are given to further illustrate the invention in more detail.

### EXAMPLE 1

Pharmaceutical composition of MFP 14 in form of vials for parenteral administration

| | | |
|---|---|---|
| Lyophilised Recombinant MFP 14 | mg | 0.5 |
| obtained according to PCT/EP/00 03003 | | |
| Sterile Saline Solution | ml | 2 |

### EXAMPLE 2

Pharmaceutical composition of MFP14 and Ubiquitin in form of vials for parenteral administration

| | | |
|---|---|---|
| Lyophilised Recombinant MFP 14 | mg | 0.5 |
| obtained according to PCT/EP/00 03003 | | |
| Ubiquitin | mg | 1 |
| Sterile Saline Solution | ml | 2 |

### EXAMPLE 3

Clinical Tests

Two female patients, 60 and 64 year old, respectively, affected by primary Parkinson for 10 years the first one, and 8 years the second one, third and fourth Hoehn and Yhar scale degrees, respectively, were treated.

Both patients received Levodopa treatment which was continued during treatment with 1 mg of recombinant MFP 14 for five consecutive days followed by 2 mg daily subsequently.

After about 20 days, the first patient showed a decrease in bradykinesia; while the second patient showed a decrease in the OFF effects and could walk with only one support (whereas she could hardly walk with double support before treatment).

1. A method of treatment of patients affected by Parkinson's disease comprising the administration to patients in need of such treatment of a therapeutically active dose of MFP 14 or active fragment thereof.

2. A method according to claim 1 wherein MFP 14 is selected from recombinant MFP 14 or MFP extracted from goat liver.

3. A method according to claim 1 further comprising the administration of Ubiquitin.
